(19) United States
(12) Patent Application Publication
(10) Pub. No.: US 2003/0084463 A1
(43) Pub. Date: May 1, 2003

(54) Method for the production of a protein

(75) Inventor: Jan Pieter Willem Vermeiden, Vreeland (NL)

Correspondence Address: BROWDY AND NEIMARK, P.L.L.C., 624 NINTH STREET, NW, SUITE 300, WASHINGTON, DC, 20001-5303, US

(73) Assignee: Leja Research B.V., Nieuw Vennep (NL)

(21) Appl. No.: 10/209,969

(22) Filed: Aug. 2, 2002

Related U.S. Application Data
(63) Continuation of application No. PCT/EP01/01217, filed on Feb. 5, 2001.

(30) Foreign Application Priority Data
Feb. 4, 2000 (EP) EP 00200395.2

(51) Int. Cl. A01K 67/027; C12N 15/85

(52) U.S. Cl. 800/7; 800/15; 435/455

(57) ABSTRACT

Described is a method for the production of a heteromeric protein comprising a first and a second peptide chain, wherein the first peptide chain is expressed in an animal, and the second peptide chain in another animal. The peptide chains are isolated from the animals and combined, resulting in formation of a functional protein. Optionally, both animals are mated, leading to offspring animals expressing both first and second peptide chains and producing the protein.

### CROSS-REFERENCE TO RELATED APPLICATION

This is a continuation application of PCT/EP01/01217 filed Feb. 5, 2001, which PCT application claims priority of European patent application number 00200395.2 filed Feb. 4, 2000, both herein incorporated by reference.

### FIELD OF THE INVENTION

The invention relates to a method for the production of a protein, the protein comprising a first and a second peptide chain, the second chain being different from the first, the peptide chains being associated to each other.

### BACKGROUND OF THE INVENTION

In the art, proteins of the above-mentioned type are known. For biological activity, association of the two different peptide chains is a prerequisite. The first and second peptides may originate from the same gene, e.g. in the case of insulin, but are usually encoded by different genes, such as is the case for antibodies and the protein hormones LH (lutenising hormone), FSH (follicle stimulating hormone), HCG (human chorion gonadopropine) and TSH (thyroid stimulating hormone). The latter all comprise an identical alpha chain and a hormone specific beta chain. It is to be understood that the protein according to the invention may comprise more than two different peptide chains or several identical peptide chains, as long as biological function of the protein requires association of two at least different peptide chains. Such proteins are known as heteromeric proteins. In case the protein consists of two different peptide chains, such a protein is also known as a heterodimeric protein. Further examples of such proteins are e.g. all other heterodimeric biological active proteins like hergulin, integrins, activins and inhibins.

In this specification, a “peptide” is defined as a single, optionally glycosylated, amino acid chain, of which the molecular weight is not defined. Such peptide will be capable to associate with at least one other peptide to form the heteromeric protein. The peptide preferably comprises an amino acid sequence of at least 15 amino acids.

In the art, the proteins of the above-mentioned type are either produced from unmanupulated animals, as is the case for HCG that is isolated from urine of pregnant human individuals, and LH and FSH, being isolated from the urine of postmenopausal females. Another way to produce such proteins is by genetic modified cells, wherein both the first and second peptide chains are expressed, properly processed and associated to each other, forming the functional protein. At present, LH, FSH and HCG are prepared by genetically modified Chinese hamster ovarium cells (CHO).

EP-A-0 404 458 describes bacterial phage vectors comprising DNA-sequences encoding ovine FSH polypeptide subunits. It is proposed therein to express the subunits in a unicellular organism and to assemble these subunits subsequently in vitro.

However, the yield of genetically engineered proteins from cell lines or unicellular organisms is low, the isolation methods are difficult, resulting in a relative expensive protein product.

In the art, it is also known to produce genetically engineered proteins through transgenic animals. E.g. Nuijens, J. H. et al, J. Biol. Chem. 28:272 (13); 8802-7, 1997 describes the production of lactoferin from the milk of transgenic mice. The problem of the production of proteins by transgenic animals is the fact that the proteins, once expressed, may biologically be active in the animal and may result in undesired side-effects. Especially in the case of the production of protein hormones, e.g. those as described above, the transgenic animal may suffer from the expression of the said proteins. In genetic modified animals, producing a genetically engineered protein in the milk, about one percent of the expressed products appeared to “escape” from the milk into the body of the animal. In order to keep the transgenic animals in a healthy condition, high expression of the protein, i.e. of the peptides constituting with said protein, is therefore limited.

### SUMMARY OF THE INVENTION

In order to obviate the above-mentioned drawbacks, the present invention relates to a method for the production of a protein, the protein comprising a first and a second peptide chain, the second chain being different from the first, the peptides chains being associated to each other, comprising the following steps:

bringing a first nucleic acid sequence, comprising

a first coding region, coding for the first peptide chain, and

a first control region, operably linked to the first coding region, controlling the expression of the first peptide chain in a first animal,

into cells of the first animal and allowing expression of the first peptide chain in the first animal by the said cells, the first peptide chain being exogenous for the first animal,

isolating the first peptide chain from the animal, or its offspring,

providing the second peptide chain in isolated form,

forming the protein by bringing together the first peptide chain isolated from the first animal with the second peptide chain under conditions allowing association of both peptide chains.

By the said method, only one of the first or second peptide chains is produced by the animal, so that a functional protein cannot be formed, allowing high expression levels of the said peptide chain. The peptide chain is exogenous for the said animal; this means that the DNA coding for the said peptide chain is a DNA molecule that has been introduced into the animal by a process such as transformation, transfection, electroporation, gene bombardment or any other method known in the art. It should be noted that it is possible that the host cell into which the said DNA-molecule has been inserted may itself also naturally harbour sequences encoding the same or a similar peptide, which is encoded by the natural genome of the said animal. For example, when a specific animal, e.g. a cow, is used in this invention, it is recognised that the said animal naturally contains, in its genome, one or more sequences encoding e.g. the alpha or beta chain of LH, FSH, CG or TSH, or the light and heavy antibody chains. A molecule such as this is referred to as an “endogenous” DNA-molecule, encoding an endogenous peptide.

The exogenous nucleic acid sequence, also referred to as the transgenic sequence, that is to be introduced into cells of a specific animal, e.g. a cow, is preferably not of cow origin. However the transgenic sequence may encode a peptide that is also endogenously produced by the animal.

The methods for bringing a nucleic acid sequence into an animal are known (see e.g. for a review Chan AWS, Cloning 1: 25-46, 1999), as well as the isolation of the transgenic products i.e. peptides, proteins (Niemann H et al, Transgenic Res. 8:237-47, 1999). According to the invention, at least one of the peptides of the (glyco)protein can thus be produced in a convenient manner with high yields. It is also possible, in case of the production of a protein, comprising more than two different peptide chains, to have the more than one of the said peptide chains produced by a single transgenic animal, as long as at least one of the peptide chains that are necessary for a functional protein product is not encoded on the transgenic nucleotide sequence, therewith avoiding any possible formation of a protein that is harmful to the animal. Preferably said nucleic acid sequence is stably introduced in the cells. This means that the transgenic nucleotide sequence is properly maintained, replicated and segregated during the cell cycle. Stable transformation may occur by chromosomal integration or by an extrachromosomal element, such as a suitable plasmid vector. Preferably the transgenic the nucleotide sequence is integrated in the genome of the animal cells.

The invention relates in particular to the production of heteromeric proteins using transgenic animals producing one or more peptides of the protein, wherein the expression of the protein as such would be harmful to the animals used, when the protein would be expressed to a level higher than the level of the natural counterpart of the said protein in the said animal. Among such proteins heteromeric enzymes and, in particular, the heteromeric protein hormones and antibodies are to mentioned.

In case a single transgenic animal is to produce more than one transgenic peptide, the sequence encoding said peptides may be located on a single nucleic acid, or on different transgenic sequences.

“Formation of the protein”, as well as “reconstitution” as used herein refers to combining the different peptide chains of a protein under conditions that allow proper association and folding of the peptides into the protein, preferably resulting in a biologically functional protein. It shall be clear that the protein is formed in vitro when the peptides are isolated from the animal before they are combined.

The skilled person will be aware of the fact that a transgenic peptide, produced according to the method of the present invention in an animal may, despite the fact that the said peptide is exogenous for the said animal, associate with a second peptide, endogenously produced by the said animal. In case such association leads to a biological active hybrid protein, care has to be taken that such hybrid protein does not lead to the above-mentioned drawback within the transgenic animal.

The transgenic peptide, produced according to the method of the present invention may also be a mutein that is capable to associate with its natural or also mutated counterpart, leading a biologically functional heteromeric mutein.

The transgenic peptide or peptides can be isolated from the animal and be combined with the second peptide or the missing peptide(s) respectively to reconstitute the protein, resulting in a functional product. The second peptide chain may e.g. be produced by tissue culture or be obtained from natural sources.

However, it is highly preferred to have the second peptide chain produced by a second transgenic animal in a way as is described above. Thus in a preferred embodiment, the present invention relates to a method as described above, further comprising the following steps:

bringing a second nucleic acid sequence, comprising

a second coding region, coding for the second peptide chain, and

a second control region, operably linked to the second coding region, controlling the expression of the second peptide chain in a second animal,

into cells of the second animal, and allowing expression of the second peptide chain in the second animal by the said cells, the second peptide chain being exogenous for the second animal,

isolating the second peptide chain from the second animal, or its offspring, and

forming the protein by bringing together the first peptide chain isolated from the first animal or its offspring, with the second peptide chain isolated from the second animal or its offspring under conditions allowing association of both peptide chains.

In this way, both peptide chains can be produced in high amounts, be isolated from the respective animals or their offspring, isolated and be brought together to reconstitute the protein.

It must be understood that the “first animal” and “second animal”, as used herein, are different individuals; the first and second animals must not be of a different species, although this may be possible if desired (e.g. in view of the required glycosilation patterns that may be required for one or more of the transgenic peptide chains).

It is preferred to at least partially purify the different peptides before the formation (reconstitution) of the protein by bringing together the said peptides. Purification of the peptides may lead to a better formation of the protein in vitro and to improved yields. Most preferably, both peptides are highly purified before the formation of the protein.

Preferably the nucleic acid sequences are brought into the animals in such a way that these sequences are propagated with the offspring of the said animals. In this specification, “offspring” means all animals, naturally bred or cloned, having the transgenic animal wherein the transgenic nucleic acid has originally been introduced as ancestor. The technique of producing transgenic animals that are capable of producing transgenic offspring is known in the art (Chan AWS, supra). Hereto, the transgenic sequences are incorporated in germ line cells of the transgenic animal. This is highly advantageous, as in that use the transgenic sequences are propagated, and the production of the protein is not limited to a single individual transgenic animal. Therefore, the method of the invention also encompasses the production of the protein from peptides, isolated from the offspring of the animal, wherein the transgenic nucleic acid sequence has originally been introduced.

In another embodiment of the invention, obviating the problem of suffering of the transgenic animals from expressing proteins, that are harmful to the said animal, the first and second animals are mated, so that their offspring is capable of producing the heteromeric protein. According to the invention, a tissue or organ with which the protein to be expressed harmfully interacts, is removed from the said offspring. Thereto, the method according to this aspect of the invention comprises the following steps:

bringing a first nucleic acid sequence, comprising

a first coding region, coding for the first peptide chain, and

a first control region, operably linked to the first coding region, controlling the expression of the first peptide chain in a first animal,

into at least germ line cells of the first animal so that the first nucleic acid sequence is propagated with the offspring of the first animal,

bringing a second nucleic acid sequence, comprising

a second coding region, coding for the second peptide chain, and

a second control region, operably linked to the second coding region, controlling the expression of the second peptide chain in a second animal,

into at least germ line cells of the second animal so that the second nucleic acid sequence is propagated with the offspring of the second animal,

producing at least one offspring animal by mating the first animal with the second animal, the said offspring animal expressing both peptide chains and forming the protein,

isolating the thus formed protein from the said offspring animal,

wherein the protein formed in the offspring animal is harmful to the said animal by interacting with a distinct tissue or organ in the offspring animal, wherein the said tissue or organ is removed from the offspring animal.

In this embodiment, the protein is formed in vivo and enables the production of transgenic animals that produce the complete transgenic heteromeric protein. The parent transgenic animals are will substantially be healthy individuals, as the parent animals express one or more, but not all peptides, necessary for the functional protein. The parent lines can be bred without any drawbacks, whereas crossing both parent lines will lead to the offspring producing the desired protein, possibly resulting in limitation of their well-being to a certain extent. The offspring animals may however produce the desired protein in high levels, without the need for any reconstitution step. As such, a method wherein peptide chains of heteromeric proteins are expressed in different parent animals, which animals are mated so that the offspring expresses the heteromeric proteins is known from e.g. WO 99/35241 and from Greenberg et al., PNAS-USA (1991) 88, pp 8327-8331.

However, the desired protein may be harmful to the offspring animals in such extent that survival of the said offspring is not possible or unethical. In case the negative effect is caused by an interaction between the protein and a distinct tissue or organ of the offspring animal, the negative effect may be obviated according to the invention by removing the said tissue or organ from the animal, if such removal still enables survival of the animal. The tissue or organ is preferably removed directly after birth or shortly thereafter. In particular, the production of gonadotropins such as LH, FSH and CG (chorion gonadotropine) can be produced according to this embodiment of the invention, when the gonads of the offspring animal are removed. E.g, a female transgenic FSH producing offspring animal can be produced that is able to survive in a healthy state upon removal of the ovaries. Similarly, healthy TSH producing offspring animals can be producing upon removal of the thyroid gland.

In a preferred embodiment of the method according to the invention, the protein is purified after formation thereof in order to provide the protein product of high purity that can be used in e.g. medicaments.

In a preferred embodiment, at least the first peptide chain is expressed by milk producing cells of a mammal and excreted into the milk by the said mammal. When the transgenic peptide(s) is present in the milk, the said peptide can easily be obtained from the mammal by milking the mammal. When both peptides (and optionally additional peptides in case the protein consists more than two different peptides) are produced in milk of different mammals, formation of the protein may take place by just mixing the milk under appropriate conditions and ratio. However, advanced methods may be used for proper protein formation; such methods are known in the art.

For expression of the required peptide chain by milk producing cells of the transgenic mammal, it is advantageous for the respective control region to comprise a casein promoter. The casein promoter has been proven to be very suitable for this purpose, although other regulatory sequences can also be used (Brink, M F et al, Theriogenolgy 53: 139-48, 2000; Whitelaw C B et al, Transgenic Res. 1: 3-13, 1991). For optimal expression, the control region preferably comprises controlling sequences, such as e.g. promoter, enhancer, ribosomal binding site etc., preferably derived from the same species as the respective transgenic animal. Thus it is preferred to incorporate a bovine casein promoter in the control region, when the peptide product is to be produced by a cow in her milk. However, it is very well possible to use a promoter of another mammalian species. It is also possible to use the bovine casein promoter in other species like mouse and rabbit.

In another preferred embodiment of the invention, the first coding region codes for the alpha chain of LH, FSH, HCG or TSH, and the second coding region for the corresponding beta chain of LH, FSH, HCG or TSH, respectively, or vice versa. By producing the alpha chain of the said peptide hormones in the one animal and the beta chain in the other, these peptide hormones can easily be produced in high quantities. Preferably the alpha chain and beta chain of the said hormones are of human origin to be optimally suitable for use in medicaments.

For veterinary medicaments, the peptide chains of the protein preferably originate from the animal species for which the medicament is intended.

Preferably, at least one, preferably both, of the coding regions is accommodated in a genomic sequence. In numerous cases, proper expression of the peptides of the protein to be reconstituted require their natural genomic environment, such as intervening sequences (introns) and/or 3′ or 5′ untranslated sequences. It is for example shown that transgenic mice, containing cDNA for the complete FSH only produce a low amount of FSH (Greenberg et al., PNAS, USA (1991), 88, p. 8327/8331).

According to the invention, preferably a nucleic acid vector is used, comprising:

a coding region, coding for a first peptide chain of a protein, the protein comprising a first and a second peptide chain, the second chain being different from the first, the peptides being associated to each other, and

a control region comprising a casein promoter, operably linked to the coding region, controlling the expression of the first peptide in an animal,

the vector being void of a sequence resulting in the expression of the second peptide chain in the said animal.

Such a vector can be used to produce the transgenic animal according to the invention. It has however to be avoided that the said vector results in expression of both first and second peptide chains, which may lead to the above-mentioned drawbacks of a transgenic animal producing high level of functional protein.

In a preferred embodiment, said nucleic acid vector comprises the coding region for the alpha or beta chain of LH, FSH, HCG or TSH, preferably from human origin. The vector can be a DNA or RNA vector as is known in the art. The coding region is preferably of genomic origin and preferably accommodated in a genomic sequence.

According to the invention animals can be obtained preferably a mammal, comprising in multiple cells, the first nucleic acid sequence according to the invention, the coding region thereof coding for an exogenous peptide. Preferably the nucleic acid sequence is propagated to the offspring of the said animal. By the presence of the said nucleic acid sequence, the exogenous peptide may be expressed in the animal and isolated for e.g. protein reconstitution purposes. In this specification, “animal” is to be understood as a higher animal, preferably a vertebrate, more preferably a mammal. Most preferred are mammals of which milk is easily isolated, i.e. cows, goats, sheep, rabbits etc.

The skilled person, aware of the above teaching will be capable of producing any protein such as a protein hormone, comprising different peptide chains that are needed for biological activity of the protein. Further, the choice of suitable animals can be easily assessed by the skilled person. In case the peptide to be produced needs glycosilation, an animal can be chosen that produces the said transgenic peptide with the required glycosilation pattern, or the glycosilation can be carried out in vitro after isolating the said peptide from the animal.

The invention will now be further explained in the following example, not being intended to limit the scope of the invention.

### EXAMPLES

The isolation, cloning and screening techniques used in the examples were done according common practise in the art; reference is made to Maniatis et al., Molecular cloning: A laboratory manual. Cold Spring Harbour Laboratory Press, Cold Spring Harbour, N.Y., USA.

1. Preparation of Transgenic Mice

a) Transgenic mice comprising transgenic DNA encoding the human alpha subunit of FSH

A mini-gene of the alpha subunit of human FSH is present between the BamHI and BglII restriction sites (Matzuk and Boime, 1988, J. Cell Biol 106, 1049-1058). This fragment contains the fused exons 2 and 3 as a cDNA sequence, linked to the intron between exon 3 and 4, and exon 4, which contains 3′-sequences. This fragment was isolated after BamHI/BglII digestion and ligated into a similarly cut transfer vector. Using site-directed mutagenesis and specific primers a ClaI site was added 5′ of the BamHI site and a NotI site was added 3′ of the BglII site. From the resulting vector the mini-gene was cut by a ClaI/NotI digestion. The active fragment of the bovine alpha S1-casein gene promoter can be obtained as a 6.3 kb NotI-ClaI fragment (Bijvoet et al., Human Molecular Genetics, 7, 1815-1824, 1998). The two fragments were isolated and ligated, using a three-point ligation into the NotI-digested and dephosphorylated cosmid pWE15 (Bijvoet et al., Human Molecular Genetics, 7, 1815-1824, 1998). After excision with NotI, this results in a linear expression cassette ready for microinjection into the pronuclei of fertilised mouse oocytes, and the generation of mice transgenic for alpha-FSH. This vector system has been used earlier for the targeted expression of acid-glucosidase (Bijvoet et al., Human Molecular Genetics, 7, 1815-1824, 1998).

b) Transgenic mice comprising transgenic DNA encoding the human beta subunit of FSH

A 3.7 kb fragment containing exon I, II, and III of the beta subunit of human FSH is present between the HindIII and BamHI restriction sites in a genomic 16.5 kb insert. The 16.5 kb insert is present in a human genomic DNA library in the phage 1MG3 (J L Keene et al., J. Biol. Chem. 264, 4769-4775, 1989). The exons in the 3.7 kb genomic sequence contain 540 -untranslated sequence, the entire coding region of beta FSH, and 1.1 kb of 3′-untranslated sequence.

The 3.7 kb fragment was isolated after HindIII/BamHI digestion and ligated into a similarly cut transfer vector. Using site-directed mutagenesis and specific primers a ClaI site was added 5′ of the HindIII site and a NotI site was added 3′ of the BamHI site. From the resulting vector the 3.7 kb fragment was cut by a ClaI/NotI digestion. The active fragment of the bovine alpha S1-casein gene promoter can be obtained as a 6.3 kb NotI-ClaI fragment (Bijvoet et al., Human Molecular Genetics, 7, 1815-1824, 1998). The two fragments were isolated and ligated, using a three-point ligation into the NotI-digested and dephosphorylated cosmid pWE15 (Bijvoet et al., Human Molecular Genetics, 7, 1815-1824, 1998). After excision with NotI, this results in a linear expression cassette ready for microinjection into the pronuclei of fertilised mouse oocytes, and the generation of mice transgenic for beta FSH. This vector system has been used earlier for the targeted expression of acid-glucosidase (Bijvoet et al., Human Molecular Genetics, 7, 1815-1824, 1998).

The isolated DNA was micro-injected into the pronuclei of fertilised mouse oocytes (CBA/BrA×C57B16) and transferred to foster mothers using standard procedures (B. Hogan et al., Manipulating the mouse embryo: A laboratory manual. Cold Spring Harbour Laboratory Press, Cold Spring Harbour, N.Y., USA)

2. Presence and Expression of Transgenic Sequences

DNA was extracted from tail biopsies, digested with EcoRI and subjected to agarose gel electrophoesis and Southern blotting to detect transgene. A NsiI-NcoI bovine alpha S1-casein fragment was used as probe [Platenburg et al., Transgenic Res. 3, 99-108, 1994]. The copy number of the transgene was determined by Southern blot analysis on digestion with SacI and hybridization with cDNA PCR fragments comprising sequences encoding the alpha or beta subunits as probes. The band intensities were compared with those of a serial dilution of plasmid DNA. A phosphoimager was used for quantification.

In total, twelve founders were obtained. Five with the alpha chain and 7 with the beta chain. They were crossed with wild-type CBA/Br×C57B1/6 mice. The founders transmitted the transgene in a Mendelian fashion. Litter seizes were normal and littermates were indistinguishable. The copy of the transgene in the offspring ranged from 2 to 10 copies as judged from the intensity of the hybridisation signal on Southern blots from tail DNA using cDNA of the alpha chain, respectively of the beta chain.

Animals were killed by cervical dislocation. Tissue samples were embedded in tissue tek (Sakura Fine tek Europe BV) flash frozen and stored in liquid nitrogen for further analysis. Cryostat sections (6 micron) were incubated with rabbit anti-human FSH antiserum in combination with swine anti-rabbit IgG antibodies conjugated to horseradish peroxidase (Dako) and stained with diamonobenzidine (Dako). The sections were counterstained with Gill's heamatoxylin. Only sections of the lactating mammary glands were positive.

The rabbit anti human FSH was raised as follows. New Zealand white rabbits were weeky intracutaneously injected with human rFSH (Gonal F Seron) mixed with Freud adjuvant. After a statisfactory titer was reached the specifity of the antiserum was verified by an enzyme linked immunosorbent assay.

Total RNA was isolated from various tissues using the RNA-zolmethod. (RNazol: Campro Scientific) . RNA (20 μg) was separated on a 1% agarose-formaldehyde gel and transferred Hybond-N membranes. The filter was hybridised with ³²P labelled alpha chain cDNA PCR fragments. Messenger RNA of the alpha chain was only detected in the RNA of the lactating mammary gland.

Analogous to the above, the presence of mRNA of the beta chain was determined. mRNA of the beta chain was only found in the RNA isolated from the lactating mammary gland.

3. Production of Transgenic Peptides in the Mouse Milk

Mouse milk was collected from day 7 after birth. Mouse milk was collected both from transgene and from non transgene animals (controls). Milk was flash frozen and stored at minus 20° C. The presence and the quantity of the alpha and the beta chain respectively in the milk of various mice was determined with an immuno-assay with a rabbit polyclonal antibody to FSH (supra). Mice expressing 2 mg/ml of the protein in their milk were used for further breeding.

No traces of the human alpha chain or the human beta chain could be detected in the milk of control animals.

4. Purification of the Peptides from the Milk

Recombinant alpha chain was isolated from the milk. The initial purification procedure consisted of three steps. Milk fat and casein were removed by centrifugation. The whey fraction was loaded on a concanavalin A -Sepharose 4B column. Adsorbed proteins were eluted with 100 mM methyl alfa-D-mannonose pyranoside.

After concentration the proteins were loaded on a Sephadex G200 column. Samples of the eluate subjected to SDS-polyacryl-amide gel electrophoresis in the samples of the mice comprising the transgenic alpha chain, a strong band of 18 kD, the molecular weight of the transgenic product was observed.

The same procedure was followed to isolate the beta chain.

5. Reconstitution

The two fluids as obtained above, one comprising the alpha chain and one comprising the beta chain were mixed is a slightly reducing environment (6.4 mM cysteamine and 3.6 mM cystamine) at a pH of 8.7 at a final concentration of 0.02 mg/ml protein. Under these conditions the alpha and the beta chains recombine spontaneously (Huth J R et al, Endocrinology 135:911-918, 1994).

The fluid with the reconstituted recombinant hormone was concentrated. The concentrate was loaded on a Sephadex G200 column and fractionised. Samples of the eluate were subjected to SDS-polyacrylamide gel electrophoresis. A major band of 36 kD, corresponding to the molecular weight of the reconstituted transgenic product was observed.

6. Production of Human FSH by Offspring Animals

Two strains of animals were established, one strain homozygote for the alpha chain of FSH and one strain for the beta chain of FSH. These strains were cross bred. The male offspring was not further used. The females were ovary ectomized in order to prevent disturbance of the well-being of the animal by hyperstimulation of the ovaries by leaking of the intact hormone into the blood. Milk production is induced in these adult females by hormone injections. The intact reconstituted hormone was present in the milk of these animals.

The recombinant hormone was purified from the milk as described in the above paragraph 4 and concentrated. The concentrate was loaded on a Sephadex G200 column and fractionised. Samples of the eluate were subjected to SDS-polyacrylamide gel electrophoresis. A major band of 36 kD, corresponding to the molecular weight of the transgenic protein product was observed.

7. Testing of the Reconstituted Protein with Immuno-assay

The 36 kD fraction of the reconstituted recombinant hormone was tested with a monoclonal antibody to human FSH (Delfia hFSH, Wallac). This antibody reacts with the beta chain of human FSH.

According to this assay, a specific activity of about 7.000-8.000 IU human FSH/mg protein was found to be present in the fluids.

8. Biological Activity of the Protein Hormone

The biological activity was tested with a human granulosa cell assay (Foldesi I et al. Human Reprod 13:1455-60, 1998). Human granulosa cells were harvested at the time of follicular aspiration after ovarian hyperstimulation for in vitro fertilisation and cultured up to 9 days. After three pre-incubations days various doses of reconstituted recombinant hormone were added. Commercial rFSH (Gonal F, Serono the Hague), was used as standard. The addition of reconstituted recombinant hormone resulted in a dose- and time dependent increase in granulosa-lutein cell oestrogen production. The reconstituted recombinant hormone appeared to have an activity of about 8.000 IU FSH/mg protein.

What is claimed is:

1. Method for the production of a protein, the protein comprising a first and a second peptide chain, the second chain being different from the first, the peptide chains being associated to each other, commprising the following steps:
bringing a first nucleic acid sequence, comprising
a first coding region, coding for the first peptide chain, and
a first control region, operably linked to the first coding region, controlling the expression of the first peptide chain in a first animal,
into cells of the first animal and allowing expression of the first peptide chain in the first animal by the said cells, the first peptide chain being exogenous for the first animal,
isolating the first peptide chain from the animal, or its offspring,
providing the second peptide chain in isolated form,
forming the protein by bringing together the first peptide chain isolated from the first animal with the second peptide chain under conditions allowing association of both peptide chains.

2. Method according to claim 1, wherein before formation of the protein the first and second peptides are at least partially purified.

3. Method according to claim 1, wherein the protein is purified after formation thereof.

4. Method according to claim 1, wherein the first peptide chain is expressed by milk producing cells of a mammal and excreted in the milk by the said mammal.

5. Method according to claim 1, wherein the first control region comprises a casein promoter.

6. Method according to claim 1, wherein the first coding region is accommodated in a genomic sequence.

7. Method according to claim 1, comprising the following steps:
bringing a second nucleic acid sequence, comprising
a second coding region, coding for the second peptide chain, and
a second control region, operably linked to the second coding region, controlling the expression of the second peptide chain in a second animal,
into cells of the second animal, and allowing expression of the second peptide chain in the second animal by the said cells, the second peptide chain being exogenous for the second animal,
isolating the second peptide chain from the second animal, or its offspring, and p1 forming the protein by bringing together the first peptide chain isolated from the first animal or its offspring, with the second peptide chain isolated from the second animal or its offspring under conditions allowing association of both peptide chains.

8. Method according to claim 2, wherein before formation of the protein the first and second peptides are at least partially purified.

9. Method according to claim 2, wherein the nucleic acid sequences are brought into the respective animals in such a way that the said sequences are propagated with the offspring of the said animals.

10. Method according to claim 2, wherein the protein is purified after formation thereof.

11. Method according to claim 2, wherein at least the first peptide chain is expressed by milk producing cells of a mammal and excreted in the milk by the said mammal.

12. Method according to claim 2, wherein at least one of the first and second control region comprises a casein promoter.

13. Method according to claim 2, wherein the first coding region codes for the α-chain of LH, FSH, HCG or TSH, and the second coding region for the corresponding β-chain of LH, FSH, HCG or TSH, respectively, or vice versa, the α-chain and the β-chain preferably being human sequences.

14. Method according to claim 2, wherein at least one of the coding regions is accommodated in a genomic sequence.

15. Method for the production of a protein, the protein comprising a first and a second peptide chain, the second chain being different from the first, the peptide chains being associated to each other, comprising the following steps:
bringing a first nucleic acid sequence, comprising
a first coding region, coding for the first peptide chain, and
a first control region, operably linked to the first coding region, controlling the expression of the first peptide chain in a first animal,
into at least germ line cells of the first animal so that the first nucleic acid sequence is propagated with the offspring of the first animal,
bringing a second nucleic acid sequence, comprising
a second coding region, coding for the second peptide chain, and
a second control region, operably linked to the second coding region, controlling the expression of the second peptide chain in a second animal,
into at least germ line cells of the second animal so that the second nucleic acid sequence is propagated with the offspring of the second animal,
producing at least one offspring animal by mating the first animal with the second animal, the said offspring animal expressing both peptide chains and forming the protein,
isolating the thus formed protein from the said offspring animal, wherein the protein formed in the offspring animal is harmful to the said animal by interacting with a distinct tissue or organ in the offspring animal, wherein the said tissue or organ is removed from the offspring animal.

16. Method according to claim 15, the protein being a gonadotropin, wherein the gonads of the offspring animal are removed.

17. Method according to claim 15, wherein the protein is purified after formation thereof.

18. Method according to claim 15, wherein at least the first peptide chain is expressed by milk producing cells of a mammal and excreted in the milk by the said mammal.

19. Method according to claim 15, wherein at least one of the first and second control region comprises a casein promoter.

20. Method according to claim 15, wherein the first coding region codes for the α-chain of LH, FSH, HCG or TSH, and the second coding region for the corresponding β-chain of LH, FSH, HCG or TSH, respectively, or vice versa, the α-chain and the β-chain preferably being human sequences.

21. Method according to claim 15, wherein at least one of the coding regions is accommodated in a genomic sequence.

22. Offspring animal, obtainable by the method according to claim 15.
